(19) United States
(12) Patent Application Publication
(10) Pub. No.: US 2006/0014181 A1
(43) Pub. Date: Jan. 19, 2006

(54) Detection of DNA mismatches and oxidative lesions

(75) Inventors: Jaqueline K. Barton, San Marino, CA (US); Jonathan Hart, Pasadena, CA (US)

Correspondence Address: DLA PIPER RUDNICK GRAY CARY US, LLP, 4365 EXECUTIVE DRIVE, SUITE 1100, SAN DIEGO, CA, 92121-2133, US

(73) Assignee: CALIFORNIA INSTITUTE OF TECHNOLOGY, Pasadena, CA (US)

(21) Appl. No.: 11/147,805

(22) Filed: Jun. 7, 2005

Related U.S. Application Data
(60) Provisional application No. 60/577,900, filed on Jun. 7, 2004.

(51) Int. Cl. C12Q 1/68; C12P 19/34

(52) U.S. Cl. 435/6; 435/91.2

(57) ABSTRACT

The present invention describes methods for directly labeling the 3′-phosphate end at a nucleotide site. Further, as internal 3′-phosphate termini on DNA duplexes are also associated generally with oxidative lesions, these methods provide a general strategy for labeling, and therefore, detecting the frequency of oxidative DNA lesions. The present invention also discloses labeling methods using terminal transferase or nontemplated DNA polymerization, where the use of either of these activities affords tagging a site, after removal of the 3′-phosphate, with polynucleotide tails. Such polynucleotide tails in turn can function as primer binding sites for use in PCR in gene analyses.

This application claims priority from U.S. Provisional Application, Ser. No. 60/577,900, filed Jun. 7, 2004, the entire contents of which is incorporated herein by reference.

### GOVERNMENT SUPPORT

This invention was made in part with government support under Grant No. NIH RO1 GM33309 awarded by the National Institutes of Health. The government has certain rights in this invention.

### BACKGROUND OF THE INVENTION

1. Field of the Invention

The invention relates generally to modification of nucleic acid sequences and more specifically, to identification of internal 3′-phosphate termini in nucleic acid duplexes.

2. Background Information

Genomic DNA varies significantly from individual to individual. Many human diseases arise from genomic variations. The genetic diversity among individuals explains the heritable differences observed in disease susceptibility. Diseases arising from such genetic variations include Huntington's disease, cystic fibrosis, Duchenne muscular dystrophy, and certain forms of breast cancer. Each of these diseases is associated with single gene mutations. Diseases such as multiple sclerosis, diabetes, Parkinson's, Alzheimer's disease, and hypertension are much more complex and may be due to polygenic or multifactorial causes. On the other hand, many of the variations in the genome are benign. The ability to scan the human genome to identify the location of genes which underlie or are associated with the pathology of such diseases is a powerful tool.

Several types of sequence variations, including restriction fragment length polymorphisms (RFLPs), short tandem repeats (STRs), variable number tandem repeats (VNTRs), insertions, and deletions result in genomic diversity. Single base pair differences, referred to as single nucleotide polymorphisms (SNPs), are the most frequent type of variation in the human genome (occurring at approximately 1 in 10³ bases). A SNP is a genomic position at which at least two or more alternative nucleotide alleles occur at a relatively high frequency (greater than 1%) in a population.

Some SNPs occur in protein-coding sequences, in which case, one of the polymorphic forms may give rise to the expression of a defective or other variant protein and, thereby causing a genetic disease. Examples of genes in which polymorphisms within coding regions result in genetic disease include beta globin (sickle cell anemia) and CFTR (cystic fibrosis). Other SNPs occur in non-coding regions. Some of these polymorphisms may also give rise to defects in protein expression (e.g., as a result of defective splicing). Still others have no effect on phenotype. Because SNPs are relatively stable (i.e., exhibit low mutation rates) and single nucleotide variations can be responsible for inherited traits, SNPs are well suited for the study of sequence variation.

Polymorphisms can be detected using microsatellite-based analysis, genetic linkage strategies and use of genetic markers to infer chromosomal locations of genes contributing to complex traits, such as type I diabetes.

Variations can also exist due to generation of DNA damage, and has been shown to be an important factor in human disease, including carcinogenesis and Parkinson's disease. Further, organisms are constantly exposed to oxidative stressors that may be involved is disease development, such as UV light exposure, mercury exposure, and development of adducts by exposure to oxidative stressors such as N-diethylnitrosamine and N-nitrosourea.

Although substantial progress has been made in identifying the genetic basis of many human diseases, current methodologies used to develop this information are limited by prohibitive costs and the extensive amount of work required to obtain genotype information from large sample populations. These limitations make identification of complex gene mutations contributing to disorders such as diabetes extremely difficult.

Some of these problems were overcome by the development of PCR based microsatellite marker analysis. Other types of genomic analysis are based on the use of markers which hybridize with hypervariable regions of DNA having multiallelic variation and high heterozygosity. The variable regions which are useful for fingerprinting genomic DNA are tandem repeats of a short sequence referred to as a minisatellite. Polymorphism is due to allelic differences in the number of repeats, which can arise as a result of mitotic or meiotic unequal exchanges or by DNA slippage during replication.

The most common used method for genotyping involves the use of Weber markers. Weber markers exhibit high polymorphisms and are therefore useful for identifying individuals in paternity and forensic testing as well as for mapping genes involved in genetic disease. In this method, generally 400 markers are used to scan each genome using PCR. PCR products can be identified by their position on a gel, and the differences in length of the products can be determined by analyzing the gel. One problem with this type of analysis is that “stuttering” tends to occur, causing a smeared result making the data difficult to interpret and score.

Other more recent advances use gene chip systems (e.g., Affymetrix HuSNP Chip™). However, such methods are expensive and time-intensive.

### SUMMARY OF THE INVENTION

Described herein are methods for directly labeling the 3′-phosphate end associated with photocleavage at a nucleotide site. Because internal 3′-phosphate termini on DNA duplexes are also associated generally with oxidative lesions, these methods provide a general strategy also for labeling and therefore detecting the frequency of oxidative DNA lesions. Labeling using terminal transferase or nontemplated DNA polymerization is also envisaged, where using either of these activities it is possible to tag a damaged site, after removal of the 3′-phosphate, with polynucleotide tails. Such polynucleotide tails in turn can be used as primer binding sites for use in PCR.

In one embodiment, a method of detecting internal 3′-phosphate termini in a nucleic acid duplex from at least one is envisaged including contacting the nucleic acid duplex with an agent to convert an internal 3′-phosphate termini to 3′-hydroxyl termini, extending 3′-hydroxyl termini present in the duplex by non-template dependent DNA polymerization, amplifying the extended product of the resulting products and identifying a nucleotide sequence-dependent feature in the resulting amplified products, where the identified feature in amplified products correlates with the presence of internal 3′-phosphate termini.

Further, the converting step may include, but is not limited to, contacting the internal 3′-phosphate termini with T4-polynucleotide kinase (T4-TNK) and the nucleic acid duplex containing a mismatched or damaged base. Moreover, the method includes, but is not limited to, contacting the duplex with an AP lyase (e.g., APN1), and in a related aspect, the non-template polymerization is carried out with TAQ polymerase, terminal deoxynucleotide transferase (TdT), or DNA polymerase Mu (Pol μ).

In a related aspect, the feature is molecular weight, length, or nucleotide sequence. Further, the feature may be determined by, but no limited to, a chromatographic method including column chromatography and electrophoresis.

In another related aspect, annealed nucleic acids may be obtained from more than one sample and nicks may be generated in the annealed product with an agent that cleaves mismatched or damaged nucleotides to generate internal 3′-phosphate termini. Moreover, at least one of the sample nucleic acid duplexes may include an annealed nucleic acid probe.

In one aspect, the agent is a hindered intercalating compound of the formula Rh(R₁)(R₂)(R₃)³⁺, where R₁ and R₂ are each independently aryl, heteroaryl, substituted aryl or substituted heteroaryl of 1 to 5 rings, and R₃ is a group of the formula

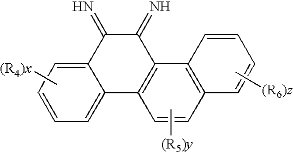

wherein x and z are each independently an integer from 1 to 4 and y is an integer from 1 to 2, and R₄, R₅, and R₆ are each independently H—, halo, HO—, H₂N—, CN—, O₂N—, HS—, O₃S—, O₃SO—, —COOH, —CONH₂, R, RO—, RNH—, RₐR_{b}N—, RO₃S—, RO₃SO—, —COOR, —CONHR, or —CONRₐR_{b}, where R, Rₐ, and R_{b} are each independently lower alkyl, cycloalkyl, lower alkenyl, lower alkynyl, or phenol, or two R₄, R₅, or R₆ together form a fused aryl ring, wherein the compound intercalates between bases in the presence of polynucleotide damage or error and does not intercalate between bases in the absence of damage or error.

In a related aspect, the agent is Δ- or Λ-Rh(bpy)₂(chrysi)³⁺, where cleaving comprises photocleavage.

In another aspect, the agent is copper (I) phenanthroline, neocazinostatin, calicheamicin, dynemicin A, esperamicin, C1027, maudropeptin, bleomycin-iron (II), halogenated uracil, iron-EDTA, or iron(II)-MPE.

In a related aspect, the mismatch is allelic and may include, but is not limited to, a single nucleotide polymorphism (SNP). In another aspect, the damage is a DNA lesion from oxidative stressor exposure, ultraviolet light exposure, or adduct formation.

In a related aspect, the amplifying step is PCR, where at least one primer for PCR is poly d(T), poly d(C), poly d(A), or poly d(G). For example, a method where the polymerase is TdT, and at least one substrate is dGTP, is envisaged. In a further related aspect, at least one primer is poly d(C). In another related aspect, at least one primer for PCR is envisaged to include, but is not limited to, dN-poly d(T), dN-poly (C), dN-poly d(A), or dN-poly d(G), which N is A, G, T, or C.

In one embodiment, a method of identifying mismatches in a sample nucleic acid duplex is envisaged, including producing nicks in the duplex with an agent that cleaves mismatched nucleotides to generate internal 3′-phosphate termini, extending the internal 3′-phosphate termini by non-template dependent DNA polymerization, amplifying the extended product, and determining a nucleotide sequence-dependent feature of the resulting amplified products, where differentiation of the feature between amplified products correlates with the presence of a mismatched base.

In another embodiment, a kit is envisaged including, but not limited to, a hindered intercalating compound, an agent for converting internal 3′-phosphate termini to internal 3′-hydroxyl termini, at least one DNA polymerase exhibiting non-template dependent polymerization activity, a set of poly d(T), poly d(C), poly d(A); and poly d(G) primers or a set of dN-poly d(T), dN-poly (C), dN-poly d(A), and dN-poly d(G) primers, wherein N is A, G, T, or C, instructions containing method steps for practicing identifying an SNP marker, identifying internal 3′-phosphate termini, or labeling a region in a nucleic acid duplex containing at least one mismatched or damaged base, or combination thereof, and a container comprising the above reagents and ancillary buffers/reagents necessary for carrying out the aforementioned methods. In a related aspect, the kit may include a label, including, but not limited to, ³²P, ³³P, ³⁵S, biotin, digoxigenin, fluorescein tetraethyl-rhodamine, TAMRA, dabcyl, or dideoxynuclotidetriphosphate.

In one embodiment, a method of labeling a nucleic acid duplex containing a mismatched base is envisaged, including contacting the nucleic acid duplex with a hindered intercalating compound, photocleaving the duplex at intercalated mismatched sites, converting internal 3′-phosphate termini generated by the photocleaving to internal 3′-hydroxyl termini; and linking the converted 3′-hydroxyl termini with a label via non-template dependent DNA polymerization.

Further, a marker identified by these method is envisaged, wherein the marker is associated with a disease selected from the group consisting of obesity, autoimmune disorders, diabetes, cardiovascular disease, central nervous system disorders, and cancer.

Exemplary methods and compositions according to this invention, are described in greater detail below.

### BRIEF DESCRIPTION OF THE DRAWINGS

FIG. 1. The general procedure for the repair and labeling of 3′ phosphate terminated DNA.

FIG. 2. A schematic overview of compounds producing 3′-phosphate terminated DNA, their repair by PNK and labeling by either polymerase or terminal transferase.

FIG. 3. Schematic of the detection of an SNP with labeling at the cleavage site.

FIG. 4. Data showing both SNP detection by end labeling (top) and by site labeling.

FIG. 5. Schematic showing the procedure for detecting mismatches in genomic DNA.

FIG. 6. Data showing differential labeling of a mismatched repair proficient cell line DNA, SW620, and a deficient cell line DNA, DU145.

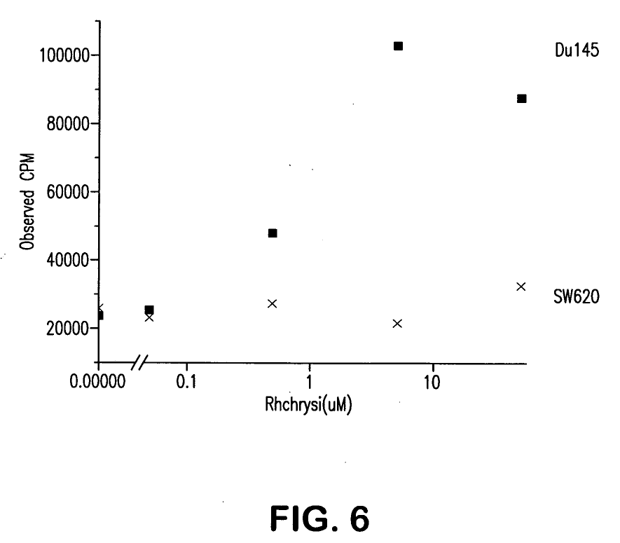

FIG. 7. Schematic representation of phosphatase assisted transferase tagging (PATT) PCR.

FIG. 8. Representative data using the PATT-PCR procedure. DNA was cleaved and then tailed with A, T, G, or C and then amplified in either the forward or reverse direction.

### DETAILED DESCRIPTION OF THE INVENTION

Before the present compositions and methods are described, it is understood that this invention is not limited to the particular methodology, protocols, and reagents described as these may vary. It is also to be understood that the terminology used herein is for the purpose of describing particular embodiments only, and is not intended to limit the scope of the present invention which will be described by the appended claims.

It must be noted that as used herein and in the appended claims, the singular forms “a”, “an”, and “the” include plural reference unless the context clearly dictates otherwise. Thus, for example, reference to “a marker” includes a plurality of such markers, reference to “a SNP” includes one or more SNPs and equivalents thereof known to those skilled in the art, and so forth.

Unless defined otherwise, all technical and scientific terms used herein have the same meanings as commonly understood by one of ordinary skill in the art to which this invention belongs. Although any methods and materials similar or equivalent to those described herein can be used in the practice or testing of the present invention, the methods, devices, and materials are now described. All publications mentioned herein are incorporated herein by reference for the purpose of describing and disclosing the nucleic acids, compounds, and methodologies which are reported in the publications which might be used in connection with the invention. Nothing herein is to be construed as an admission that the invention is not entitled to antedate such disclosure by virtue of prior invention.

Molecular probes have been described herein that bind and with photoactivation cleave DNA neighboring destabilized DNA mispairs (1,2). These probes are now described for application in single nucleotide polymorphism (SNP) discovery. Both in the context of that application as well as for the detection of mismatched DNA as a diagnostic of various cancers associated with mismatch repair deficiency, sensitive methods were developed to quantitate the frequency of mismatches. By labeling the site of mismatch photocleavage, either by fluorescence, radioactivity, or polymerization, quantitation of mismatch cleavage and hence the frequency of mismatches can be achieved. Herein described are methods for directly labeling the 3′-phosphate end associated with photocleavage at a mismatch site. Because internal 3′-phosphate termini on DNA duplexes are also associated generally with oxidative lesions, this method provides a general strategy also for labeling and therefore detecting the frequency of oxidative DNA lesions.

Photocleavage by mismatch-targeting reagents generates a 3′-phosphate terminus at the nicked DNA site neighboring the mismatch (3). The 3′-phosphate termini of DNA are typically unreactive in most enzyme catalyzed reactions (4). Additionally, in the context of an internal nick in the DNA, they are unreactive with typical phosphatases. However, certain DNA repair enzymes are able to remove these 3′-phosphate ends. T4 polynucleotide kinase, as well as hPNKp, and rat homologs, are able to perform this action (5,6,7). Once the 3′-phosphate is removed, the oxidatively cleaved DNA strand changes from unreactive to reactive with many DNA modifying enzymes. Thus, the 3′-hydroxyl terminated DNA nick once generated can be labeled using fluorescence or radioactivity with DNA polymerases, terminal transferase, nontemplated DNA polymerization, or any other method of 3′-hydroxyl DNA labeling.

In one embodiment, labeling using terminal transferase or nontemplated DNA polymerization is described. Using either of these activities it is possible to tag the damaged site, after removal of the 3′-phosphate, with a polynucleotide tail. This polynucleotide tail in turn can be used as a primer binding site for use in PCR. If another primer binding site exists on the opposite strand of DNA, the location of the damage relative to the known primer site can be determined. In this way a much more straight forward alternative to LM-PCR (9) or TD-PCR (10) can be obtained.

This methodology can be extended beyond labeling of damaged ends near mismatches to obtain a general labeling scheme for oxidative DNA lesions that produce a 3′-phosphate terminated DNA. Sugar oxidation at any position on the sugar ring results in the formation of some 3′-phosphate terminated DNA. Nucleic acid base damage can be converted chemically to 3′-phosphate terminated DNA by treatment with hot piperidine (12). Additionally, if T4-PNK is exchanged with APN1, a yeast AP lyase that can cleave not only 3′-phosphates but also 3′-phosphoglycolates, should result in more efficient labeling results (13).

As used herein, the term “internal 3′-phosphate termini,” including grammatical variations thereof, means an internal nick in duplex DNA due to hydrolysis of a phosphodiester bond between two bases within a polynucleotide chain, resulting in an 3′-phosphate terminus and a 5′ hydroxyl terminus within the chain.

As used herein, the term “non-template dependent DNA polymerization,” including grammatical variations thereof, means the formation of biological nucleic acid polymers without the requirement of template nucleotides for processivity. In a related aspect, non-template dependent polymerization includes, but is not limited to, the activities of TAQ polymerase (Brownstein M J, et al., Biotechniques (1996) 20(6):1004-6, 1008-10), terminal deoxynucleotide transferase (TdT) (Belyavsky A, et al., Nucleic Acids Res (1989) 17(8):2919-32), and DNA polymerase Mu (Pol μ) (Dominguez O, et al., EMBO J (2000) 19(7):1731-42).

As used herein, the term “nucleotide sequence-dependent feature,” including grammatical variations thereof, means any physicochemical property of a polymer consisting essentially of nucleotide bases. For example, such features include, but are not limited to, base composition, G-C richness, A-T percentage, motif/element sequences, length, complementary base pair formation, Cₒt, Rₒt, molecular weight, total charge, fragmentation to daughter ions from ionization, and nucleotide sequence. In a related aspect, “contrasting a nucleotide sequence-dependent feature,” or “identifying a nucleotide sequence dependent feature” including grammatical variations thereof, means ascertaining, comparing, or distinguishing with respect to differences between physicochemical properties of nucleic acid containing polymers.

In one embodiment, a chromatographic method is used, including any technique, analytical or preparative, for separating the components of a mixture by differential adsorption of compounds to absorbents, partition between stationary and mobile immiscible phases, ion exchange, or a combination of these to include, but not limited to, adsorption, affinity, affinity-elution, ampholyte-displacement, argentation, ascending, bio-specific elution, charge transfer chromatography, circular, countercurrent, covalent, descending, dye-ligand, electro, exclusion, frontal, gas, gas-liquid, gel filtration, gel-permeation, high performance liquid affinity, high performance liquid, high pressure liquid, hydrophobic, ion-exchange, ionic interaction, ion-moderated partition, ligand mediated, liquid, liquid-liquid, metal-chelate affinity, molecular exclusion, molecular sieve, negative, paper, partition, permeation, positive, pseudo-affinity, reverse-phase, salting-out, sievorptive, steric-exclusion, subunit exchange, thermal-elution, thin layer, and triazine dye.

As used herein, the term “mismatched,” including grammatical variations thereof, means the occurrence of a base in one polynucleotide strand of a duplex nucleic acid that is not complementary to the corresponding base in the second polynucleotide strand.

As used herein, the term “annealed nucleic acid probe,” including grammatical variations thereof, means a renatured, heat-denatured nucleic acid, which renaturation results in duplex formation by controlled cooling. In a related aspect, a probe includes, but is not limited to, an oligo- or polynucleotide that is complementary to an oligonucleotide or nucleic acid sequence, where the probe may or may not comprise a label, and where hybridization of the probe to a sequence of interest can be used in a method of detecting that sequence.

As used herein, the term “allelic,” including grammatical variations thereof, means one of several alternative forms of a gene occupying a given locus on a chromosome.

As used herein, the term “single nucleotide polymorphism (SNP),” including grammatical variations thereof, means the occurrence of single base variations in the genetic code that occur about every 1000 bases along the human genome.

As used herein, the term “marker,” including grammatical variations thereof, includes reference to a locus on a chromosome that serves to identify a unique position on the chromosome. A “polymorphic marker” includes reference to a marker which appears in multiple forms (alleles) such that different forms of the marker, when they are present in a homologous pair, allow transmission of each of the chromosomes in that pair to be followed. A genotype may be defined by use of one or a plurality of markers.

As used herein “amplifying,” including grammatical variations thereof, means the construction of multiple copies of a nucleic acid sequence or multiple copies complementary to the nucleic acid sequence using at least one of the nucleic acid sequences as a template. Amplification systems include the polymerase chain reaction (PCR) system, ligase chain reaction (LCR) system, nucleic acid sequence based amplification (NASBA, Cangene, Mississauga, Ontario), Q-Beta Replicase systems, transcription-based amplification system (TAS), and strand displacement amplification (SDA). See, e.g., Diagnostic Molecular Microbiology: Principles and Applications, D. H. Persing et al., Ed., American Society for Microbiology, Wash., D.C. (1993). The product of amplification is termed an amplicon.

“PCR” or “Polymerase Chain Reaction” a technique for the synthesis of large quantities of specific DNA segments, consists of a series of repetitive cycles (Perkin Elmer Cetus Instruments, Norwalk, Conn.). Typically, the double stranded DNA is heat denatured, the two primers complementary to the 3′ boundaries of the target segment are annealed at low temperature and then extended at an intermediate temperature. One set of these three consecutive steps is referred to as a cycle.

The process utilizes sets of specific in vitro synthesized oligonucleotides to prime DNA synthesis (e.g., poly d(N) primers, where N is A, G, C, or T). The design of the primers is dependent upon the sequences of DNA that are desired to be analyzed. The technique is carried out through many cycles (usually 20-50) of melting the template at high temperature, allowing the primers to anneal to complementary sequences within the template and then replicating the template with DNA polymerase.

The products of PCR reactions can be analyzed by separation in agarose gels followed by ethidium bromide staining and visualization with UV transillumination. Alternatively, radioactive dNTPs can be added to the PCR in order to incorporate label into the products. In this case the products of PCR are visualized by exposure of the gel to x-ray film. The added advantage of radiolabeling PCR products is that the levels of individual amplification products can be quantitated.

As used herein, the term “primer,” including grammatical variations thereof, means an oligonucloeitde required as the starting point for the stepwise synthesis of a polynucleotide from mononucleotides by the action of a nucleotidyltransferase.

In a related aspect, dN-poly d(A), dN-poly d(G), dN-poly d(T), or dN-poly d(C) are envisaged as primers for PCR, where the primers allow for controlled annealing near the site of non-template polymerization at 3′-hydroxyl termini.

As used herein, the term “ionized fragment,” including grammatical variations thereof, means a daughter molecular entity where there has been a loss of one or more electrons from a neutral chemical parent species, which molecular entity can be detected by a mass spectrometry means.

As used herein, the term “agent for converting internal 3′-phosphate termini to internal 3′-hydroxyl,” including grammatical variations thereof, means an chemical entity that can dephosphorylate a 3′-phosphate terminus of a polynucleotide. For example, T4 polynucleotide kinase (T4-PNK) is an enzyme which can catalyze such a reaction.

In one embodiment, ancillary buffers/reagents are chemical compositions which provide the necessary conditions to allow for a reaction to occur. For example, T4 ligase buffer provides the proper ionic and pH conditions for T4-PNK to remove a phosphate group from an internal 3′-phosphate terminus, and as such would be included as an ancillary buffer/reagent in a kit.

The term “hindered intercalating compound” or “hindered intercalating agent” as used herein refers to a compound that is not capable of substantially intercalating between the bases of a normal duplex polynucleotide, but is capable of intercalating between the bases of a duplex polynucleotide having error and/or damage. A labeled agent is a hindered intercalating agent bearing a detectable label, as defined below. A “cleaving” agent is a hindered intercalating agent that is capable of cleaving or catalyzing the cleavage of a polynucleotide duplex in which it is intercalated. A “photocleaving” compound or agent is a hindered intercalating agent capable of catalyzing photolysis of a polynucleotide in which it is intercalated.

Such hindered intercalating compounds, and their methods of preparation are described in U.S. Pat. No. 6,444,661, the contents of which is incorporated herein in its entirety.

The terms “damage” and “error” as used herein refer to a departure from the “normal” or ideal structure of a polynucleotide duplex. In the “ideal” structure, all bases are paired with complementary bases, and no nicks, breaks, or gaps occur in the backbones. “Error” describes the condition in which a base is paired with a non-complementary base, or a base is absent from a position (abasic), or a gap exists in the sequence of one strand (e.g., the strands have different numbers of bases, and the unpaired location does not occur at the end of the strand). “Error” includes simple base pair mismatches, for example in which a denatured DNA sample is hybridized with a substantially (but not completely) complementary oligonucleotide probe: the probe and target can depart from complentarity by one or more bases. “Damage” describes the condition in which the conformation of the duplex is perturbed, for example by a nick in the backbone, T-T dimerization, and the like.

In humans, each cell division requires the replication of approximately six billion bases of DNA. Most errors are detected and corrected by DNA repair enzymes. However, DNA repair enzymes are inactive or inefficient in some forms of cancer: these cancers can be diagnosed by the presence of higher than normal numbers of base mismatches per cell. A “condition” or “disorder” characterized by polynucleotide damage or error is a pathological state that can be distinguished from a normal state by the presence of an increased level, rate, or concentration of damage and/or errors in polynucleotide duplexes. The increase in polynucleotide damage and/or error can be determined with respect to a control, or with respect to a known or previously measured rate established for “normal” individuals.

The term “effective amount” refers to the amount of compound necessary to cause cleavage of an oligonucleotide duplex having a base mismatch when subjected to light of sufficient energy. The minimum effective amount can vary depending on reaction conditions and the identity of the bases involved in the mismatch, but in general will range from a ratio of about 100:1 to about 1:1 nucleotide:compound. The effective amount for a particular application can vary with the conditions employed, but can be determined using only routine experimentation.

The term “label” as used herein refers to a moiety that is detectable or can be manipulated to provide a detectable signal. Suitable detectable labels include, without limitation, radioactive atoms such as ³H, ¹⁴C, and the like, fluorophores, chromophores, electron-dense reagents, isotopic labels, enzymes capable of catalyzing signal reactions such as chromogenic, luminescent, and fluorescent reactions, binding ligands, cleaving molecules, and the like. “Binding ligands” are moieties capable of binding a labeled compound or a solid support; for example, a detectable label can comprise a moiety capable of binding a polynucleotide duplex to a solid support, where the polynucleotide can be detected directly, for example by PCR or hybridization assays. Alternatively, a binding ligand can bind to another compound which bears a detectable label, for example an enzyme-labeled antibody. Cleaving molecules are capable or cleaving, or catalyzing the cleavage of, polynucleotides: this can serve as a label by, for example, releasing one end of a duplex polynucleotide from a surface-bound complex. One can detect the released ends, for example by end-labeling the strands prior to cleavage, or can detect the newly cleaved end bound to the support, for example where the duplexes are end-protected prior to cleavage, and subject to enzymatic degradation in the absence of the end protecting group.

The term “cleavage conditions” refers to reaction conditions sufficient to cause cleavage of an oligonucleotide duplex having a base mismatch in the presence of an effective amount of a compound of the invention. “Photocleavage conditions” are those conditions sufficient to cause photolysis of a polynucleotide in the presence of an effective amount of photocleaving compound or agent.

The term “mutagenic agent” refers to a physical, chemical, or biological agent capable of causing DNA and/or RNA damage or errors. Examples of known mutagenic agents include, without limitation, ionizing radiation, ultraviolet light, 2-aminopurine, 5-bromouracil, hydroxylamine, nitrous acid, ethyl ethane sulfonate, nitrosamines, nitrogen mustard, acridine, proflavin, and the like.

The term “stringent conditions” refers to polynucleotide hybridization conditions (generally a combination of temperature, concentration, and denaturing agent) under which a probe oligonucleotide will bind to a target polynucleotide only if completely complementary. “Non-stringent conditions” are hybridization conditions which tolerate the presence of one or more base mismatches, i.e., where substantially complementary polynucleotides will hybridize. Substantially complementary polynucleotides can differ from exact complementarity in 5% or more of the base positions, or can contain a few as a single base mismatch.

One aspect of the invention is based on the discovery that one can prepare intercalating compounds that are too hindered to intercalate between the bases of a “normal” polynucleotide duplex, but can intercalate between the bases of a duplex in the presence of damage or error. Such compounds are useful for indicating the presence of polynucleotide damage or error, for diagnosing conditions characterized by polynucleotide damage or error, for separating or isolating damaged or erroneous polynucleotides, and for treating conditions characterized by polynucleotide damage or error.

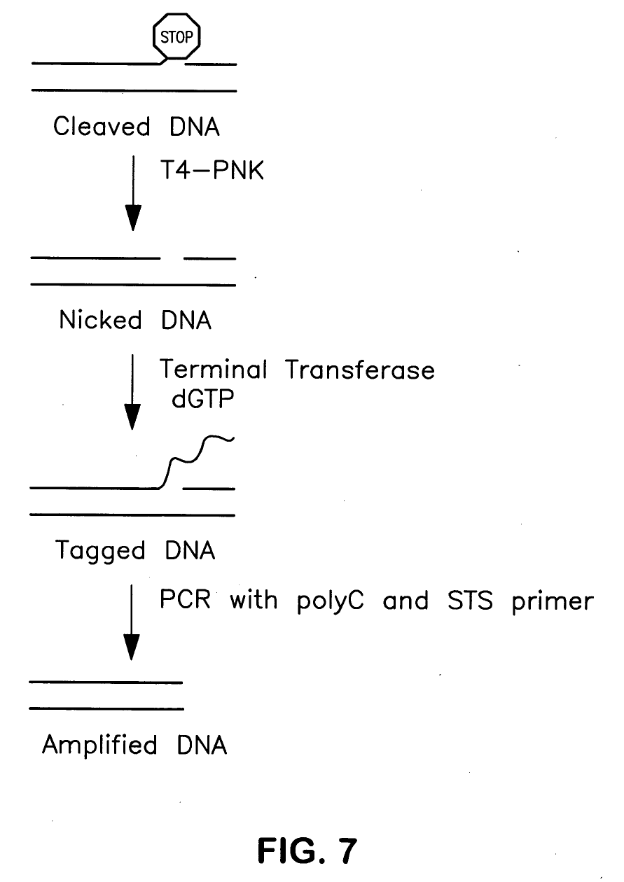

One method of the invention is a method for determining the existence of a difference between a target polynucleotide and a probe oligonucleotide. Previous methods for detecting a base mismatch between a probe and a target relied on sensitive adjustment of hybridization conditions (e.g., temperature and concentration), such that hybridization occurred only where the probe and target were completely complementary, and not otherwise (“stringent conditions”). Using hindered intercalating compounds, however, one can directly label polynucleotide duplexes having a base mismatch, and thus directly detect lack of full complementarity between a probe and a target under non-stringent conditions. Thus, in one embodiment of the invention, a sample containing a target polynucleotide is provided, contacted with a probe oligonucleotide under non-stringent conditions, contacted with a labeled hindered intercalating compound, and the product duplex nucleic acids examined for the presence of label. This method can be used, for example, to diagnose hereditary differences and/or the presence of genetic defects, to distinguish between different strains of pathogenic organisms, to establish paternity, to distinguish between a subject's DNA and DNA found in a forensic sample, amongst other uses (e.g., see FIG. 7, differential labeling between cell lines showing differences in mismatch repair). The sample/target polynucleotide can be provided in single strand or double strand form, but is preferably denatured prior to hybridization with the probe oligonucleotide. The probe oligonucleotide can be as short as about 8-10 bases, up to a length of several thousand bases: the probe can be as long or longer than the target polynucleotide.

The following examples are intended to illustrate but not limit the invention.

### EXAMPLES

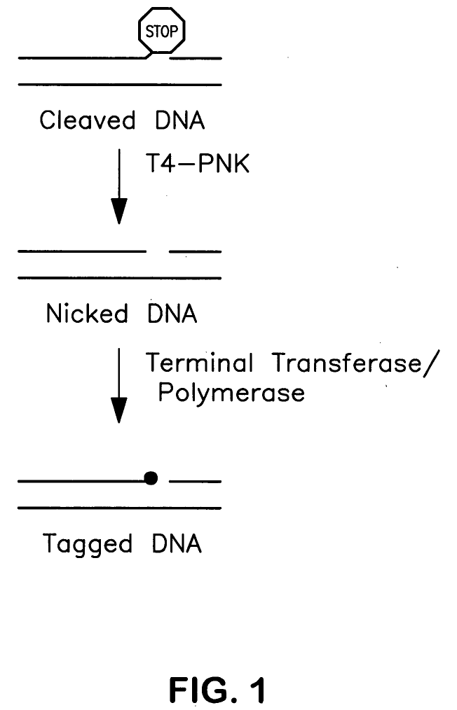

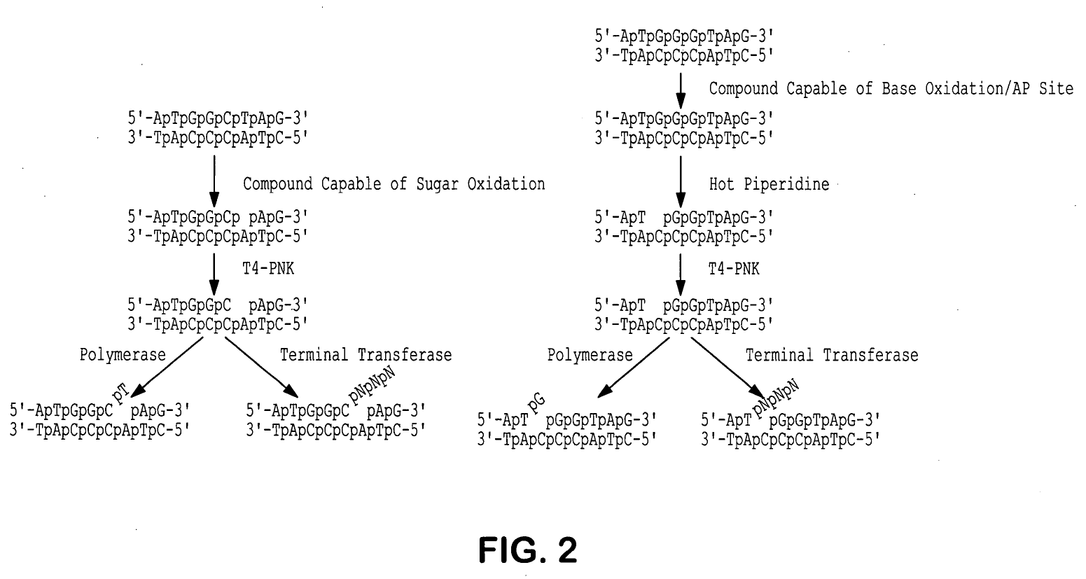

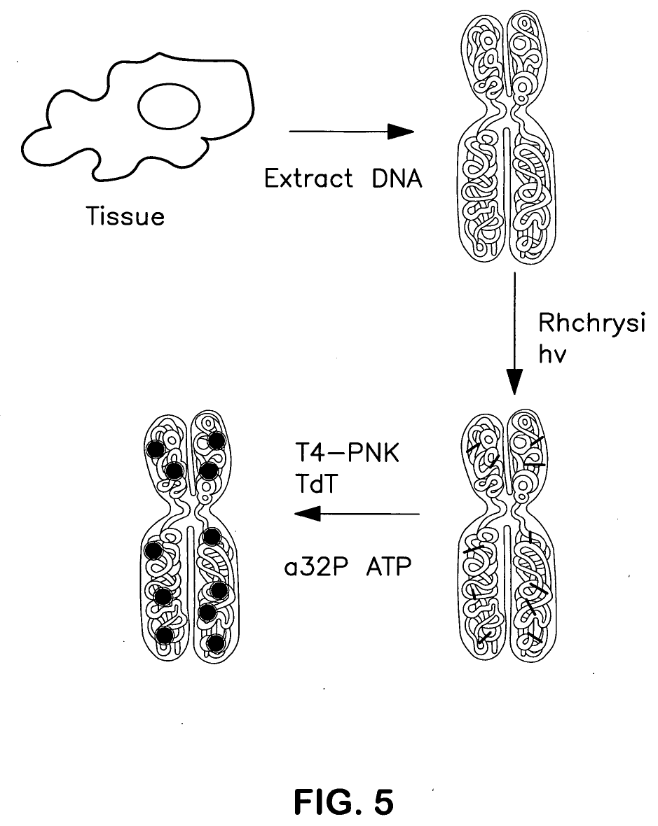

The general scheme for labeling and repair can be seen in FIG. 1. Briefly, the cleaved DNA (i.e., subsequent to binding and photocleavage with [Rh(bpy)₂(chrysi)]³⁺) is treated with T4-PNK, whereafter the resulting dephosphorylated 3′hydroxyl terminus of the nicked DNA is treated with TdT in the presence of a label, thereby transferring the label to the nicked site (e.g., FIG. 2 and FIG. 5).

While in one embodiment, [Rh(bpy)₂(chrysi)]³⁺ can be used to produce 3′-phosphate termini, other agents capable of producing such ends are listed in Table 1.

TABLE 1
Agents Capable of Generating 3′-Phosphate Termini*

| | | 3′-Phosphate |
|---|---|---|
| | 3′-Phosphate | Generation Upon |
| | Generation by | Treatment with |
| Agents | Direct Action | a Base |
| Copper (I) phenanthroline | Yes | — |
| Rhodium (III) complexes | Yes | — |
| of phi, chrysi, phzi, | | |
| phen, bpy, and | | |
| derivatives | | |
| Neocarzinostatin, | Yes | — |
| calicheamicin, dynemicin | | |
| A, esperamicin, C1027, | | |
| maudropeptin, and other | | |
| ene-diyne drugs | | |
| Bleomycin-iron (II) | Yes | — |
| Halogenated uracil | Yes | — |
| Gamma radiolysis | Yes | — |
| Iron (II)-EDTA | Yes | — |
| Iron (II)-MPE | Yes | — |
| Singlet oxygen | — | Yes |
| Hydroxyl radical | — | Yes |
| Ruthenium complexes of | — | Yes |
| phen, bpy, dppz, etc. | | |

*For a complete review see Burrows, C J and Muller, J G, Chem Rev (1998) 98: 1109-1151.

### Example 1

### Protocol Utilized in Testing the Labeling Strategy

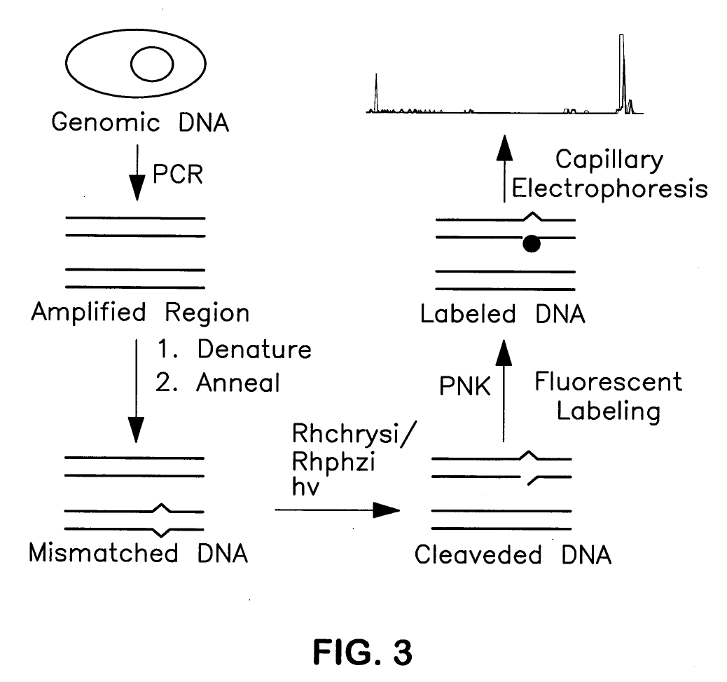

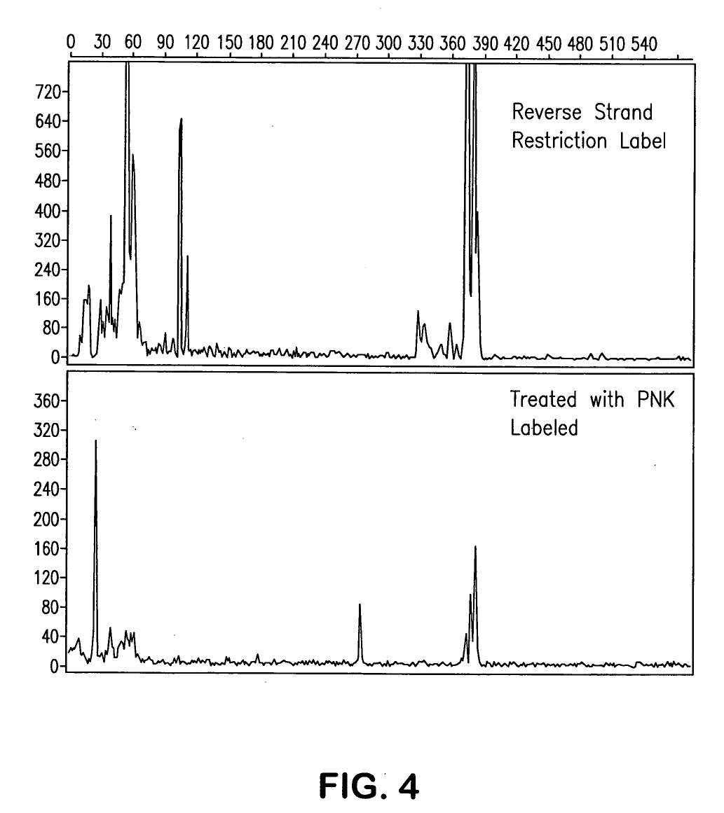

A pooled human genomic DNA sample was amplified using primers specific for the TNF promoter region, F:AGA,GAT,AGA,ACA,AAA,GGA,TAA,GGG,CTC,AG (SEQ ID NO:1) and R:GTG,TGG,CCA,TAT,CTT,CTT,AAA,CG (SEQ ID NO: 2) using Roche FastStart™ High Fidelity polymerase according to standard proceedure. After polymerization, 20U calf intestinal alkaline phosphatase and 6U exonuclease I were added. The DNA was further purified by using a QlAgen PCR cleanup column™ and eluting in 10 mM Tris pH 8.0. This DNA was denatured, by heating to 99° C. for 20 minutes and annealed, by the addition of buffer and slow cooling to room temperature, to generate a final concentration of 20 mM Tris pH 7.0 and 100 mM NaCl; this denaturation and annealing generates mismatches. These mismatches were cleaved by 1 μM [Rh(bpy)₂(chrysi)]³⁺ upon irradiation at 440 nm for 25 minutes. After cleavage 80U T4 polynucleotide kinase and 4 μL T4 ligase buffer were added to remove terminal 3′-phosphates. This mixture was then dried under reduced pressure and labeled using Applied Biosystems's SNaPshot™ kit following its procedures. The fluorescently labeled products were separated and detected using an ABI 310 prism capillary electrophoresis instrument (e.g., see FIG. 3 and FIG. 4). Under these conditions a new peak was detected 275 bases in length corresponding to a known SNP site in this sequence. Without the addition of rhodium complex or PNK no cleaved product is detected.

### Example 2

### Protocol Used in Testing phosphatase Assisted Transferase Tagging PCR (PATT-PCR)

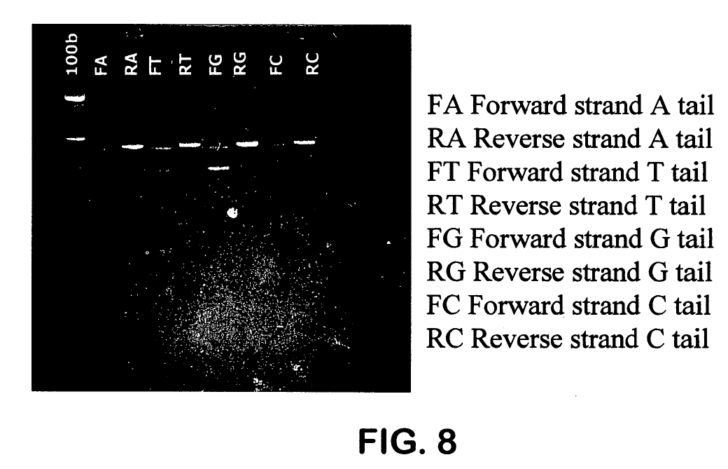

DNA was PCR amplified from two plasmids which were polymorphic at a single site, one containing the G allele, the other contained the C allele with F:CGC,GTT,GGC,CGA,TTA,ATT,AAT,G (SEQ ID NO: 3) and R: GCT,GCG,CAA,CTG,TTG,GGA,AG (SEQ ID NO:4), using Taq polymerase from Roche Biochemicals under standard conditions. After polymerization, 20U calf intestinal alkaline phosphatase and 6U exonuclease I were added. The DNA was further purified by using a QIAgen PCR cleanup column™ and eluting in 10 mM Tris pH 8.0. This DNA was denatured, by heating to 99° C. for 20 minutes and annealed, by the addition of buffer and slow cooling to room temperature, to generate a final concentration of 20 mM Tris pH 7.0 and 100 mM NaCl; this denaturation and annealing generates mismatches. These mismatches were cleaved by 1 μM [Rh(bpy)₂(chrysi)] ³⁺ upon irradiation at 440 nm for 25 minutes. After cleavage 80U T4 polynucleotide kinase and 4 μL T4 ligase buffer were added to remove terminal 3′-phosphates. The DNA was ethanol precipitated and dried under reduced pressure to remove the T4 ligase buffer. The nicks in the DNA were tagged using terminal transferase under the following conditions: 400 U recombinant terminal transferase, 200 mM potassium cacodylate, 25 mM Tris HCl, 5 μg BSA, 0.75 mM CoCl₂, 5 μM dGTP in a total volume of 20 μL pH 6.6. The reaction mixture was incubated at 37° C. for 1 hour and stopped by denaturing at 70° C. for 20 minutes. The tagged products were then amplified by Taq polymerase in two reactions under standard conditions, except with an annealing temperature of 45° C. using the primers F, as described above, and C₁₅ (SEQ ID NO: 5) or R and C₁₅ (SEQ ID NO: 5). The products of this reaction were then analyzed by agarose gel electrophoresis. Using this system, a new band is detected at approximately 290 base pairs in length (see FIG. 8), when the forward primer is used, but no new bands are seen when the reverse primer is used. This would correspond to an oxidative nick on the reverse strand at +275 bp.

All of the compositions and methods disclosed and claimed herein can be made and executed without undue experimentation in light of the present disclosure. While the compositions and methods of this invention have been described in terms of illustrative embodiments, it will be apparent to those of skill in the art that variations may be applied to the composition, methods and in the steps or in the sequence of steps of the methods described herein without departing from the concept, spirit and scope of the invention. More specifically, it will be apparent that certain agents which are both chemically and physiologically related may be substituted for the agents described herein while the same or similar results would be achieved. Although the invention has been described with reference to the above examples, it will be understood that modifications and variations are encompassed within the spirit and scope of the invention.

### REFERENCES

1. Jackson, B. A., Barton, J. K. J. Am. Chem. Soc. 119, 12986-12987 (1997); Jackson, B. A., Barton, J. K. Biochemistry 38, 4655 (1999); Jackson, B. A., Barton, J. K. Biochemistry, 39, 6176 (2000); Junicke, H., Hart, J. R., Kisko, J., Glebov, O., Kirsch, I. R., Barton, J. K. Proc. Natl. Acad. Sci. USA 100, 3737-3742 (2003).
2. U.S. Pat. No. 6,444,661 B 1: Detection and Treatment of Duplex Polynucleotide Damage, J. K. Barton, B. A. Jackson, and B. P. Hudson, Sep. 3, 2002; U.S. patent application Ser. No. 10/015,997: Method and Compositions for Detecting Polynucleotide Duplex Damage and Errors, J. K. Barton and H. Junicke, Filed Dec. 8, 2000 (See CIT Pending Application Nos. 3341).
3. Jackson, B. A., Ph.D. Dissertation, California Institute of Technology; Sitlani, A., Long, E. C., Pyle, A. M., Barton, J. K. J. Am. Chem. Soc. 114, 2302 (1992).
4. Friedberg, E. C., Walker, G. C., Siede, W. DNA repair and Mutagenesis (1995).
5. Cameron, V., Uhlenbeck, O. C. Biochemistry 16, 5120-5126 (1977).
6. Jilani, A., Ramotar, D., Slack, C., Ong, C., Yang, X. M., Scherer, S. W., Lasko, D. D. J. Biol. Chem. 274, 24176-24186 (1999).
7. Habraken, Y., Verly, W. G. FEBS Lett. 160, 46-50 (1983).
8. Gorczyca, W., Gong, J. P, Darzynkiewicz, Z. Cancer Res. 53, 1945-1951 (1993).
9. Pfeifer, G. P., Steigerwald, S. D., Mueller, P. R., Wold, B., Riggs, A. D. Science 246, 810-813 (1989).
10. Komura, J., Riggs, A D. Nucl. Acids. Res. 26, 1807-1811 (1998).
11. Pogozelski, W. K., Tullius, T. D. Chem. Rev. 98, 1089-1107 (1998).
12. Burrows, C. J., Muller, J. G. Chem. Rev. 98, 1109-1151 (1998).
13. Popoff, S. C., Spira, Al, Johnson, A. W., Demple, B. Proc. Natl. Acad. Sci USA 87, 4193-4197 (1990).

Accordingly, the invention is limited only by the following claims.

What is claimed is:

1. A method of detecting internal 3′-phosphate termini in a nucleic acid duplex from at least one sample comprising:
a) contacting the nucleic acid duplex with an agent to convert internal 3′-phosphate termini to 3′-hydroxyl termini;
b) extending 3′-hydroxyl termini present in the duplex by non-template dependent DNA polymerization;
c) amplifying the extended product of step (b); and
d) identifying a nucleotide sequence-dependent feature in the resulting amplified products,
wherein the feature in the amplified products correlates with the presence of internal 3′-phosphate termini.

2. The method of claim 1, wherein the feature is molecular weight, length, or nucleotide sequence.

3. The method of claim 2, wherein the feature is length.

4. The method of claim 3, wherein the length is determined by a chromatographic method selected from the group consisting of column chromatography and electrophoresis.

5. The method of claim 1, comprising annealing nucleic acids obtained from more than one sample and producing nicks in the annealed product with an agent that cleaves mismatched or damaged nucleotides to generate internal 3′-phosphate termini.

6. The method of claim 5, wherein at least one of the sample nucleic acid duplexes comprises an annealed nucleic acid probe.

7. The method of claim 5, wherein the agent is a hindered intercalating compound.

8. The method of claim 7, wherein the compound is of the formula Rh(R₁)(R₂)(R₃)³⁺, wherein R₁ and R₂ are each independently aryl, heteroaryl, substituted aryl or substituted heteroaryl of 1 to 5 rings, and R₃ is a group of the formula 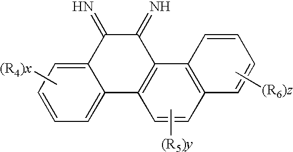
wherein x and z are each independently an integer from 1 to 4 and y is an integer from 1 to 2, and R₄, R₅, and R₆ are each independently H—, halo, HO—, H₂N—, CN—, O₂N—, HS—, O₃S—, O₃SO—, —COOH, —CONH₂, R, RO—, RNH—, RₐR_{b}N—, RO₃S—, RO₃SO—, —COOR, —CONHR, or —CONRₐR_{b}, where R, Rₐ, and R_{b} are each independently lower alkyl, cycloalkyl, lower alkenyl, lower alkynyl, or phenol, or two R₄, R₅, or R₆ together form a fused aryl ring, wherein the compound intercalates between bases in the presence of polynucleotide damage or error and does not intercalate between bases in the absence of damage or error.

9. The method of claim 8, wherein the compound is Δ- or Λ-Rh(bpy)₂(chrysi)³⁺.

10. The method of claim 9, wherein the compound is Δ-Rh(bpy)₂(chrysi)³⁺.

11. The method of claim 10, wherein cleaving comprises photocleavage.

12. The method of claim 5, wherein the agent is copper (I) phenanthroline, neocazinostatin, calicheamicin, dynemicin A, esperamicin, C1027, maudropeptin, bleomycin-iron (II), halogenated uracil, iron-EDTA, or iron(II)-MPE.

13. The method of claim 1, wherein the converting step comprises contacting the internal 3′-phosphate termini with T4-polynucleotide kinase (T4-TNK).

14. The method of claim 1, wherein the nucleic acid duplex comprises a mismatch or damaged base.

15. The method of claim 14, wherein the mismatch is allelic.

16. The method of claim 15, wherein the mismatch is a single nucleotide polymorphism (SNP).

17. A marker identified by the method of claim 1.

18. The marker of claim 17, wherein the marker is associated with a disease selected from the group consisting of obesity, autoimmune disorders, diabetes, cardiovascular disease, central nervous system disorders, and cancer.

19. The method of claim 14, wherein the damage is a DNA lesion from oxidative stressor exposure, ultraviolet light exposure, or adduct formation.

20. The method of claim 1, comprising contacting the duplex with an AP lyase.

21. The method of claim 20, wherein the AP lyase is APN1.

22. The method of claim 1, wherein non-template polymerization is carried out with TAQ polymerase, terminal deoxynucleotide transferase (TdT), or DNA polymerase Mu (Pol μ).

23. The method of claim 22, wherein the amplifying step is PCR.

24. The method of claim 23, wherein at least one primer for PCR is poly d(T), poly d(C), poly d(A), or poly d(G).

25. The method of claim 24, wherein the non-template polymerase is TdT, and at least one substrate is dGTP.

26. The method of claim 25, wherein at least one primer is poly d(C).

27. The method of claim 23, wherein at least one primer for PCR is dN-poly d(T), dN-poly (C), dN-poly d(A), or dN-poly d(G), which N is A, G, T, or C.

28. A method of identifying mismatches in a sample nucleic acid duplex comprising:
a) producing nicks in the duplex with an agent that cleaves mismatched nucleotides to generate internal 3′-phosphate termini;
b) extending the internal 3′-phosphate termini by non-template dependent DNA polymerization;
c) amplifying the extended product of step (b); and
d) determining a nucleotide sequence-dependent feature of the resulting amplified products,
wherein differentiation of the feature between amplified products correlates with the presence of a mismatched base.

29. The method of claim 28, wherein the feature is molecular weight, length, or nucleotide sequence.

30. The method of claim 28, wherein at least one strand of the duplex is an annealed nucleic acid probe.

31. The method of claim 28, wherein the agent is a hindered intercalating compound.

32. The method of claim 31, wherein the compound is of the formula Rh(R₁)(R₂)(R₃)³⁺,
wherein R₁ and R₂ are each independently aryl, heteroaryl, substituted aryl or substituted heteroaryl of 1 to 5 rings, and R₃ is a group of the formula 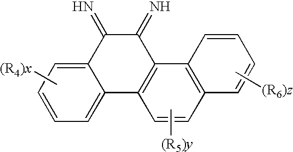
wherein x and z are each independently an integer from 1 to 4 and y is an integer from 1 to 2, and R₄, R₅, and R₆ are each independently H—, halo, HO—, H₂N—, CN—, O₂N—, HS—, O₃S—, O₃SO—, —COOH, —CONH₂, R, RO—, RNH—, RₐR_{b}N—, RO₃S—, RO₃SO—, —COOR, —CONHR, or —CONRₐR_{b}, where R, Rₐ, and R_{b} are each independently lower alkyl, cycloalkyl, lower alkenyl, lower alkynyl, or phenol, or two R₄, R₅, or R₆ together form a fused aryl ring, wherein the compound intercalates between bases in the presence of polynucleotide error and does not intercalate between bases in the absence of error.

33. The method of claim 32, wherein the compound is Δ- or Λ-Rh(bpy)₂(chrysi)³⁺.

34. The method of claim 32, wherein the compound is Δ-Rh(bpy)₂(chrysi)³⁺.

35. The method of claim 28, wherein the internal 3′-phosphate termini is contacted with T4-polynucleotide kinase (T4-TNK).

36. The method of claim 28, wherein the mismatch is allelic.

37. The method of claim 28, wherein the mismatch is a single nucleotide polymorphism (SNP).

38. A marker identified by the method of claim 28.

39. The marker of claim 37, wherein the marker is associated with a disease selected from the group consisting of obesity, autoimmune disorders, diabetes, cardiovascular disease, central nervous system disorders, and cancer.

40. The method of claim 28, wherein non-template polymerization is carried out with TAQ polymerase, terminal deoxynucleotide transferase (TdT), or DNA polymerase Mu (Pol μ).

41. The method of claim 40, wherein the amplifying step is PCR.

42. The method of claim 41, wherein at least one primer for PCR is poly d(T), poly d(C), poly d(A), or poly d(G).

43. The method of claim 42, wherein non-template polymerization is carried out with TdT, and at least one substrate is dGTP.

44. The method of claim 43, wherein at least one primer is poly d(C).

45. The method of claim 41, wherein at least one primer for PCR is dN-poly d(T), dN-poly (C), dN-poly d(A), or dN-poly d(G), which N is A, G, T, or C.

46. A kit comprising:
a) a hindered intercalating compound;
b) an agent for converting internal 3′-phosphate termini to internal 3′-hydroxyl termini;
c) at least one DNA polymerase exhibiting non-template dependent polymerization activity;
d) a set of poly d(T), poly d(C), poly d(A), and poly d(G) primers or a set of dN-poly d(T), dN-poly (C), dN-poly d(A), and dN-poly d(G) primers, wherein N is A, G, T, or C;
e) instructions containing method steps for practicing identifying an SNP marker, identifying internal 3′-phosphate termini, or labeling a region in a nucleic acid duplex containing at least one mismatched or damaged base, or combination thereof; and
f) a container comprising reagents (a)-(d) and ancillary buffers/reagents necessary for carrying out the methods of component (e).

47. The kit of claim 46, further comprising a label.

48. The method of claim 47, wherein the label is ³²P, ³³P, ³⁵S, biotin, digoxigenin, fluorescein tetraethyl-rhodamine, TAMRA, dabcyl, or dideoxynuclotidetriphosphate.

49. A method of labeling a nucleic acid duplex containing a mismatched base comprising:
a) contacting the nucleic acid duplex with a hindered intercalating compound;
b) photocleaving the duplex at intercalated mismatched sites;
c) converting internal 3′-phosphate termini generated by the photocleaving to internal 3′-hydroxyl termini; and
d) linking the converted 3′-hydroxyl termini with a label via non-template dependent polymerization.

50. The method of claim 49, wherein the compound is of the formula Rh(R₁)(R₂)(R₃)³⁺, wherein R₁ and R₂ are each independently aryl, heteroaryl, substituted aryl or substituted heteroaryl of 1 to 5 rings, and R₃ is a group of the formula 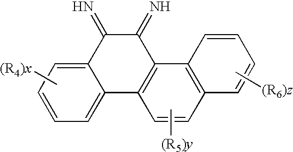
wherein x and z are each independently an integer from 1 to 4 and y is an integer from 1 to 2, and R₄, R₅, and R₆ are each independently H—, halo, HO—, H₂N—, CN—, O₂N—, HS—, O₃S—, O₃SO—, —COOH, —CONH₂, R, RO—, RNH—, RₐR_{b}N—, RO₃S—, RO₃SO—, —COOR, —CONHR, or —CONRₐR_{b}, where R, Rₐ, and R_{b} are each independently lower alkyl, cycloalkyl, lower alkenyl, lower alkynyl, or phenol, or two R₄, R₅, or R₆ together form a fused aryl ring, wherein the compound intercalates between bases in the presence of polynucleotide damage or error and does not intercalate between bases in the absence of damage or error.

51. The method of claim 50, wherein the compound is Δ- or Λ-Rh(bpy)₂(chrysi)³⁺.

52. The method of claim 51, wherein the compound is Δ-Rh(bpy)₂(chrysi)³⁺.

53. The method of claim 49, wherein the internal 3′-phosphate termini is contacted with T4-polynucleotide kinase (T4-TNK).

54. The method of claim 49, wherein the label is ³²P, ³³P, 35S, biotin, digoxigenin, fluorescein tetraethyl-rhodamine, TAMRA, dabcyl, or dideoxynuclotidetriphosphate.

55. The method of claim 49, wherein the mismatch is allelic.

56. The method of claim 55, wherein the mismatch is a single nucleotide polymorphism (SNP).

57. A marker identified by the method of claim 49.

58. The marker of claim 57, wherein the marker is associated with a disease selected from the group consisting of obesity, autoimmune disorders, diabetes, cardiovascular disease, central nervous system disorders, and cancer.

59. The method of claim 49, wherein the polymerization is carried out with TAQ polymerase, terminal deoxynucleotide transferase (TdT), or DNA polymerase Mu (Pol μ).


SEQUENCE LISTING (5 sequences)

SEQ ID NO: 1 - DNA, 29 bp, Artificial sequence
  Primer
agagatagaacaaaaggataagggctcag

SEQ ID NO: 2 - DNA, 23 bp, Artificial sequence
  Primer
gtgtggccatatcttcttaaacg

SEQ ID NO: 3 - DNA, 22 bp, Artificial sequence
  Primer
cgcgttggccgattaattaatg

SEQ ID NO: 4 - DNA, 20 bp, Artificial sequence
  Primer
gctgcgcaactgttgggaag

SEQ ID NO: 5 - DNA, 15 bp, Artificial sequence
  Primer
ccccccccccccccc